(19) United States
(12) Patent Application Publication
(10) Pub. No.: US 2019/0274551 A1
(43) Pub. Date: Sep. 12, 2019

(54) CORE BODY TEMPERATURE SYSTEM

(71) Applicant: Razzberry Inc., New Haven, CT (US)

(72) Inventor: Alexandra Barton-Sweeney, Berkeley, CA (US)

(21) Appl. No.: 16/411,521

(22) Filed: May 14, 2019

Related U.S. Application Data
(63) Continuation of application No. 15/151,909, filed on May 11, 2016, now abandoned.
(60) Provisional application No. 62/160,030, filed on May 12, 2015.

(51) Int. Cl. A61B 5/01; A61B 5/00; A61B 5/024; G16H 40/63; A61B 10/00

(52) U.S. Cl. CPC A61B 5/01; A61B 5/6833; A61B 5/024; A61B 5/0008; A61B 5/1118; A61B 10/0012; A61B 5/7275; A61B 5/7282; A61B 5/4806; G16H 40/63

(57) ABSTRACT

A system and method are provided for determining a core body temperature or a physiological function. The system and method includes using multiple sensors in combination with analytic techniques.

### CROSS-REFERENCE TO RELATED APPLICATIONS

This application claims priority to U.S. patent application Ser. No. 15/151,909 filed May 11, 2016 which claims priority to U.S. provisional application Ser. No. 62/160,030, filed May 12, 2015, the contents of which are hereby incorporated by reference in their entirety.

### BACKGROUND OF THE DISCLOSURE

The subject matter disclosed herein generally relates to a system for approximating core body temperature, and a system that uses body temperature to determine a health state, metabolic function, or fertility parameter, for example.

Core body temperature (CBT) is a vital sign. It is important to measure CBT accurately in order to diagnose, treat, and monitor a number of health conditions. If core body temperature is not measured accurately, the result may impact diagnosis and treatment as well as compromise patient health. Accurate core body temperature calculation can be used for a number of applications by establishing a baseline temperature and temperature range to be used with future recordings, for example. These applications can include patient monitoring, presence or absence of fever, fertility level, physical activity, diurnal and nocturnal activity, sleep quality, sleep architecture, metabolic function, kidney function, hormonal changes, heart function, sleepiness onset tracking, close observation in resolving hypothermia/hyperthermia, monitoring the effect of treatment for antimicrobial therapy for infection, before and during a blood transfusion to monitor for signs of a reaction, during or post operation, and for a number of diseased states, among others. CBT is also helpful in understanding sweat rate and in use with other methods to measure health, metabolic, or hormonal states. Current options for on-going temperature monitoring require subjects to wake up regularly to take internal temperature measurements, or use invasive temperature tracking techniques.

The metabolic rate regulates many bodily functions. Hormonal levels based on thyroid function are a major determinant of the metabolic rate. To determine metabolic function and metabolic rate, several blood tests, including thyroid hormone levels (T3, T4, T7, FTI, TSH, reverse T3, etc.) may be conducted. These tests only measure the quantity of these analytes at one point in time and are unable to include additional variables in the analysis, such as body temperature, age, weight, height, fat percentage, body mass index, lean body mass, gender, or menstrual cycle phase for menstruating women, for example, which can influence these levels. By not taking into account these influencing factors and an individual's metabolic needs, what may appear to fall within the “normal” range for the general population may actually be an under-functioning or over-functioning metabolic state for the individual. An under-functioning as opposed to a mal-functioning thyroid may therefore not be diagnosed using these tests.

In order to provide a better analysis of the metabolic function and the functioning of the homeostatic control system to detect a health condition, one could perform vital sign tests such as heart rate tracking and temperature tracking such as basal body temperature or core body temperature. Body temperature tracking can provide valuable information related to metabolic function, such as information about the menstrual cycle phases, as well as a number of metabolic conditions such as hypothyroidism, high cholesterol, pituitary issues, gonadal issues, adrenal issues, hypoglycemia, pancreatitis, drug or alcohol abuse, kidney, liver, and cardiovascular conditions, central nervous system abnormalities, metabolic toxicities, reproductive phase (menopause, perimenopause, puberty, etc.), fibromyalgia, and others.

Body temperature is the result of the balance between heat production and heat loss. Depending on the metabolic activity level of body tissues, the rate of heat generated and lost can fluctuate. Body temperature is therefore distributed unevenly and provides a great deal of variability. In a state of rest, heat generation is predominantly a result of the vital organs such as the heart, liver brain, and endocrine organs. Core body temperature is the temperature of the blood supplying organs such as the brain, abdominal, and thoracic cavities. Core body temperature is more stable than peripheral body temperature, the temperature of tissues such as the skin, which is more susceptible to environmental factors, however many factors including chronobiological rhythms and behavioral and environmental factors can influence these body temperatures.

An example of behavioral factor influences includes waking times. Three different chronotypes have been defined in relation to circadian research: morning, evening, and intermediate types. Depending on the time of day an individual normally rises, his/her specific circadian rhythm temperature minimums and maximums are affected. For example, “Persons categorized as “morning types” have significantly earlier nadir and peak times for temperatures than do “evening types.” (Kelly, G. “Body Temperature Variability”, Alternative Medicine Review Volume 12, Number 1 2007)

The importance of determining a normal range for a subject is critical when determining overall health and the impact of medicine on certain conditions and the tracking of conditions that have a correlation to body temperature, e.g., antibiotics for infection or fever tracking. What is normal for one individual may not be normal for another, and temperature ranges change depending on the time of day and other endogenous and exogenous factors. A lack of understanding the individual's body temperature can lead to improper treatment or diagnosis.

There are wide variations in practice for measuring body temperature. The measurement of core body temperature may seem simple, but several issues affect the accuracy of the reading, such as the measurement site, the reliability of the instrument and subject technique. True core temperature readings can only be measured by invasive means, such as placing a temperature probe into the esophagus, pulmonary artery, or urinary bladder. These sites tend to be reserved for patients who are critically ill. Other sites such as the rectum, oral cavity, axilla, temporal artery (forehead), and external auditory canal are accessible and can be used to provide an estimation of the core temperature. The temperature measured between these sites can vary greatly.

Axillary temperature is measured at the axilla (armpit) by placing the thermometer in the central position and adducting the arm close to the chest wall. There are no main blood vessels around this area, and the measurement may be affected by the environment and perspiration, for example, so this site may not provide an accurate measurement of core temperature.

Rectal temperature may be a more accurate method for measuring the core temperature, but using this site is more time consuming than other methods, and this site may result in inaccurate readings due to the presence of feces, and might be considered unfavorable for some patients. There is also low blood flow to this area, so changes to the core temperature may not be tracked immediately.

The temporal artery thermometer is quick to use. It is held over the forehead and senses infrared emissions radiating from the skin. However, its reliability and validity have not been widely tested.

The oral cavity temperature is considered to be reliable when the thermometer is placed posteriorly into the sublingual pocket, but other locations in the oral cavity can cause inaccurate temperature readings. Other factors can also affect the accuracy of oral cavity temperature such as recent ingestion of food or fluid, having a respiratory rate >18 per minute, smoking, and possibly oxygen therapy. (McCallum L, Higgins D (2012) Measuring body temperature. Nursing Times; 108: 45, 20-22)

A common oral thermometer application is in the tracking of basal body temperature (BBT). BBT is the lowest temperature attained by the body during rest. True BBT is difficult to measure with current methods since it occurs during the sleeping hours. Currently, BBT is approximated by taking the temperature, primarily oral temperature, upon waking. BBT is sensitive to a number of physiological changes, including hormonal changes, such as the hormonal changes during the menstrual cycle. Oral temperature can be influenced by many factors, such as food intake and physical activity. This is why women are instructed to awaken at the same time each morning and use a thermometer before sitting up out of bed. If she waits fifteen minutes after waking or uses the bathroom or stands up, her temperature reading could be skewed. In addition, according to a study on diurnal and nocturnal core body temperature changes in ovulating women (Lee, Kathryn A. Circadian Temperature Rhythms in Relation to Menstrual Cycle Phase, Dept. of Physiological Nursing, Univ. of Washington, Seattle, Wash., Journal of Biological Rhythms, Vol. 3, No. 3, 1988), “it is during the early morning hours that rapid-eye-movement sleep predominates and thermoregulation is impaired. Therefore, BBT measurements immediately upon awakening are more likely to reflect sleep stage and ambient temperature than the thermogenic properties of progesterone. Additionally, user-generated BBT charts based on morning measurements can be difficult to interpret, even by high qualified specialists.” (McCallum L, Higgins D (2012) Measuring body temperature. Nursing Times; 108: 45, 20-22) This is especially true since there are more factors than circa mensal rhythms that can influence readings. This outlines the need for a non-invasive, automatized data analysis based on a sophisticated data model.

There have been some attempts to develop wearable thermometers, such as temperature “patches” for non-invasive determination of body temperature. The patch form factor has been preferred because of a desire for immobility and close adhesion to the skin. Patch prototypes describe usage under or near the armpit, which is known to be inaccurate for estimating core body temperature due to variations in perspiration and arm position, requiring the user to firmly keep his/her arm at the side, which is difficult to do for an extended period of time, and especially during illness, sleep, and daily activity. Another issue involves the need to place a new patch in the exact same location for the temperature readings in order to have ongoing tracking between patches because skin temperature can vary widely depending on where on the body it is taken. Placement in the exact same location may be difficult. Skin temperature degrees by themselves cannot be correlated to core body temperature. Current temperature patch prototypes do not take other factors into account, instead they make use of one or more thermal inputs while looking for a rise or fall in relation to a degree threshold.

Body temperature will change according to endogenous and exogenous factors, including many rhythms, such as the circadian, circamensal, and circannual rhythms Diurnal and nocturnal temperatures change according to known patterns, which result in shifts between the core body temperature in relation to the temperature of the extremities. For example, with the onset of sleep, core body temperature decreases, while the temperature of the extremities increase, and vice versa during diurnal activity.

A problem with current non-invasive methods is they do not provide enough information to determine the individual's normal and abnormal, or healthy and unhealthy temperature ranges for peripheral or core body temperatures nor do they take into account other factors that influence body temperature beyond the exact temperature data.

Heat balance and thermoregulatory control are important to measure and track in many situations. For example, heat balance in perioperative and post-operative patients is critical yet difficult to track. Ineffective thermoregulation is common, resulting in a propensity toward hypothermia and heat imbalances as a result of several factors, including the effects of neuromuscular blocking drugs, such as general and regional anesthesia, intravenous fluids, and environmental stress.

There is a need for a non-invasive temperature tracking approach that can accurately estimate core body temperature, mean body temperature, and basal body temperature and track changes over time.

### BRIEF DESCRIPTION OF THE DISCLOSURE

The inventors hereof have developed a method to use temperature readings to predict core body temperature or other physiological functions based on external body sensors. The described method allows for the use of temperature tracking in the diagnosis, treatment, and monitoring of a number of conditions, including fertility, kidney function, metabolic function, fever, infection, and others.

A method to determine a physiological function result, comprising: determining the temperature of a first body location on a body to obtain a first body temperature value; optionally determining the temperature of a second body location on a body to obtain a second body temperature value; optionally determining the ambient temperature to determine an ambient temperature value; obtaining a physiological function result from a predetermined prediction equation, wherein the obtaining comprises a comparison of the amplitude of the temperature values, the mesor of the temperature values, the mean of the temperature values, the peak of the temperature values, the nadir of the temperature values, or the acrophase of the temperature values, or a combination comprising one or more of the foregoing is provided.

A system for determining a health state, comprising: two or more sensors, each sensor comprising: a membrane configured to be applied to an external surface of a body of a subject; one or more temperature monitors configured to detect and/or measure a temperature, each monitor located within the sensor in thermal contact with the membrane; optionally, a transmitting apparatus configured to transmit the output from the one or more temperature monitors; a power source configured to apply power to the one or more temperature sensors and the transmitting apparatus; optionally, an ambient environmental monitor configured to detect and/or measure an environmental condition in a vicinity of a subject; a processing unit configured to receive and process signals produced by the temperature monitors and optional ambient environmental monitor, to determine time-dependent parameters of temperature change, and to calculate a core body temperature is also provided.

A kit for determining a health state, comprising: one or more sensors, each sensor comprising: a membrane configured to be applied to an external surface of a body of a subject; one or more temperature monitors configured to detect and/or measure a temperature, each monitor located within the sensor in thermal contact with the membrane; a transmitting apparatus configured to transmit the output from the one or more temperature monitors; a power source configured to apply power to the one or more temperature sensors and the transmitting apparatus; optionally, an ambient environmental monitor configured to detect and/or measure an environmental condition in a vicinity of a subject; instructions for using the one or more sensors, the instructions comprising access to a processing unit configured to receive and process signals produced by the temperature monitors and optional ambient environmental monitor, to determine time-dependent parameters of temperature change, and to calculate a core body temperature is also provided.

The provided method and system can be used in several ways, as described further herein. In one embodiment, a temperature sensor is placed at two or more different locations on the subject's body, where at least one sensor represents the distal temperature, where the sensor is closer to the body's extremities based on its location on the skin; and at least one sensor represents the proximal temperature, where the sensor is closer to the body's core. By calculating the difference of the skin temperature at the distal sensor and the proximal sensor, the distal to proximal temperature gradient can be calculated.

The distal to proximal temperature gradient can be used to approximate core body temperature by combining the distal to proximal gradient with additional information, such as expectations for diurnal and nocturnal temperature measurements, the distal to proximal temperature ratios in relation to cyclic rhythm parameters, such as a circadian rhythm, sleep-wake cycles, activity rhythms, circannual rhythm, circa mensal rhythm, or a combination comprising one or more of the foregoing circadian rhythms, sleepiness onset calculations, rhythm strength, de-synchronized or synchronized traits, heart rate, vasomotion, heat-loss balance and distribution, expected shifts for diurnal or nocturnal activity, as well as changes in temperature mesor, temperature acrophase, and temperature amplitude. This additional information can be based on historical data of the subject or population data, obtained using sources such as a database of obtained data, or other sources, as known in the art. This information can be combined with the distal to proximal temperature gradient in a predetermined prediction equation to obtain a physiological function result, as described further herein.

In an embodiment, one or more of the acrophase (the peak time of a rhythm from the cosine curve best fitting the data), MESOR (Midline Estimating Statistic of Rhythm—the value midway between the highest and lowest values of the (cosine) function best fitting to the data), and amplitude (the difference between the maximum height of a wave and the rhythm-adjusted mean of the wave form) are used to analyze the data obtained, including the temperature data.

In an embodiment, a system to measure heat balance comprising two or more sensors to evaluate vasomotion based on skin-surface temperatures is described. Based on sensor placement on specified parts of the body, such as during surgery, this system can be used to determine peripheral compartment temperatures and regional body heat distribution. This data can be used to approximate mean and regional body temperature. It can be used for example in the peri- and post-operative setting to monitor neuromuscular activity to avoid hypothermia and track the impact of surgery and neuromuscular blocking drugs on the body.

In an embodiment, the temperature amplitude at a single or multiple temperature sensors can be used. The temperature amplitude can be used to determine basal body temperature changes, for example. This allow for the use of disposable patches where the location of the patch, the temperature readings, or the sensors themselves, may be inconsistent from patch to patch. By using values other than exact degrees, inconsistencies such as placement, calibration, sensor issues, sweat rate, or ambient temperature changes can be minimized to obtain a more accurate physiological function result, such as an estimate of body temperature.

Changes in the amplitude of the temperature values, the mesor of the temperature values, the mean of the temperature values, the peak of the temperature values, the nadir of the temperature values, or the acrophase of the temperature values, or a combination comprising at least one of the foregoing over time, for example, can be used to predict a fertility parameter, physiologic, or a metabolic function parameter. Focusing on amplitude rather than actual temperature measurements allows for improved estimates of fertility by removing error associated with non-hormonal influencers on temperature and the need to compare exact invasive temperature measurements with each other Amplitude, mesor, and acrophase changes between nightly temperature readings occur during different menstrual phases. Mesor increases and amplitude is lowered during the post-ovulatory/luteal phase, in comparison to the pre-ovulatory phase. This information can be used in the predetermined prediction equation, along with the amplitude, mesor, or acrophase values obtained, and the predetermined prediction equation can predict when ovulation will occur or has occurred, and at what point a woman is in her menstrual cycle. This allows the challenges of using exact temperature measurements to approximate BBT to be overcome, and also eliminates the need to approximate core body temperature in order to approximate cycle phase and fertility level. The use of amplitude changes for example, can also be applied to other conditions where amplitude shifts have been shown to occur, for example diminished amplitudes have been seen in advanced cancer patients, poorly physically fit individuals, elderly patients, patients experiencing a manic depressive episode, and chronically ill patients such as those suffering from HIV, among others.

The method described here can be used to overcome the challenges of using skin temperature as a non-invasive predictor of core body temperature, heat balance, thermoregulation, vasomotion, and BBT.

The method provided uses temperature sensors in varying locations on the body or on a device to gather information about the skin temperature as well as ambient temperature. This data is then combined with mathematics, bioinformatics, and/or artificial intelligence to estimate core body temperature or to diagnose and track various health conditions.

By including ambient and skin temperatures, the endothermic process of the subject being measured can be analyzed. The endothermic process describes a process or reaction in which the system maintains a metabolically favorable temperature, largely by the use of heat set free by its internal bodily functions in relation to ambient temperatures. The thermoneutral zone is a range of environmental temperatures in which the metabolic rate is low and independent of temperature. The basal metabolic rate is the metabolic rate of a resting animal at a temperature within the thermoneutral zone. Within the thermoneutral zone, the body temperature is regulated by altering heat loss through the skin. Below the lower critical temperature, the animal produces metabolic heat to compensate for increased heat loss to the environment. Above the upper critical temperature, the animal must expend energy to lose heat by panting or sweating, which makes its metabolic rate increase. Below or above the thermoneutral zone, the subject's metabolic rate increases. By taking into account external temperatures, one can incorporate the endothermic processes in the estimation of core body temperature, basal body temperature, heat balance and distribution, vasomotion, and basal metabolic rate, and obtain information about the metabolic function.

These and other advantages and features will become more apparent from the following description taken in conjunction with the drawings.

### BRIEF DESCRIPTION OF DRAWINGS

The subject matter which is regarded as the disclosure, is particularly pointed out and distinctly claimed in the claims at the conclusion of the specification. The foregoing and other features, and advantages of the disclosure are apparent from the following detailed description taken in conjunction with the accompanying drawings in which:

FIG. 1 is a top view of a core body temperature device in accordance with an embodiment of the invention;

FIG. 2 is a side sectional view of the core body temperature device of FIG. 1;

FIG. 3 are plots comparing the time of distal and proximal skin temperatures, heart rate and CBT;

FIG. 4 is a table of circadian temperature rhythm parameters as a function of menstrual cycle;

FIG. 5 is a table of salivary progesterone levels at two phases of the menstrual cycle;

FIG. 6 is a plot of the mean time course of subjective sleepiness, temperature gradient and CBT;

FIG. 7 are plots of salivary melatonin, temperature gradient, skin temperature, CBT and Karolinska sleepiness scale as a function of time; and

FIG. 8 are plots of menstrual cycle phase and temperature rhythms as a function of time.

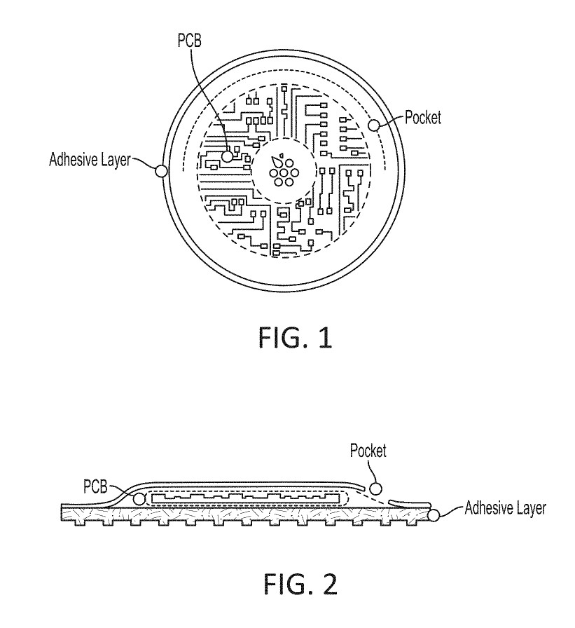

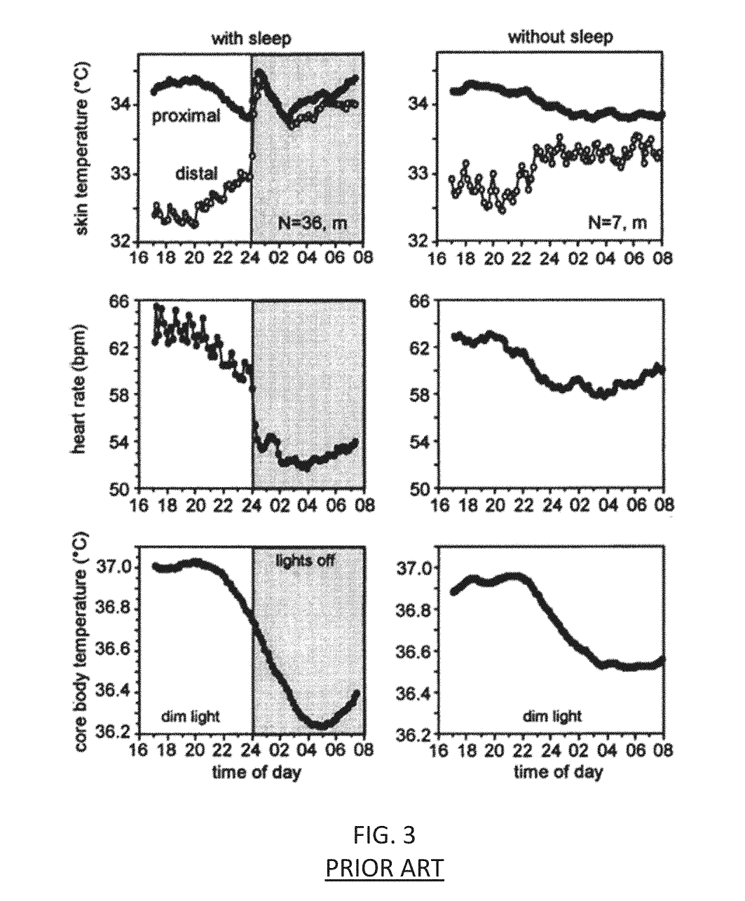

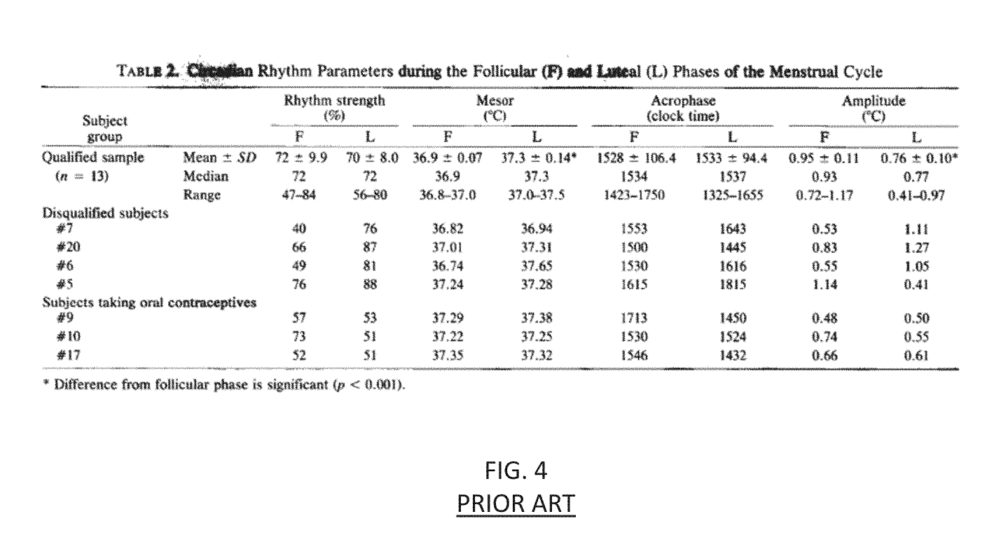

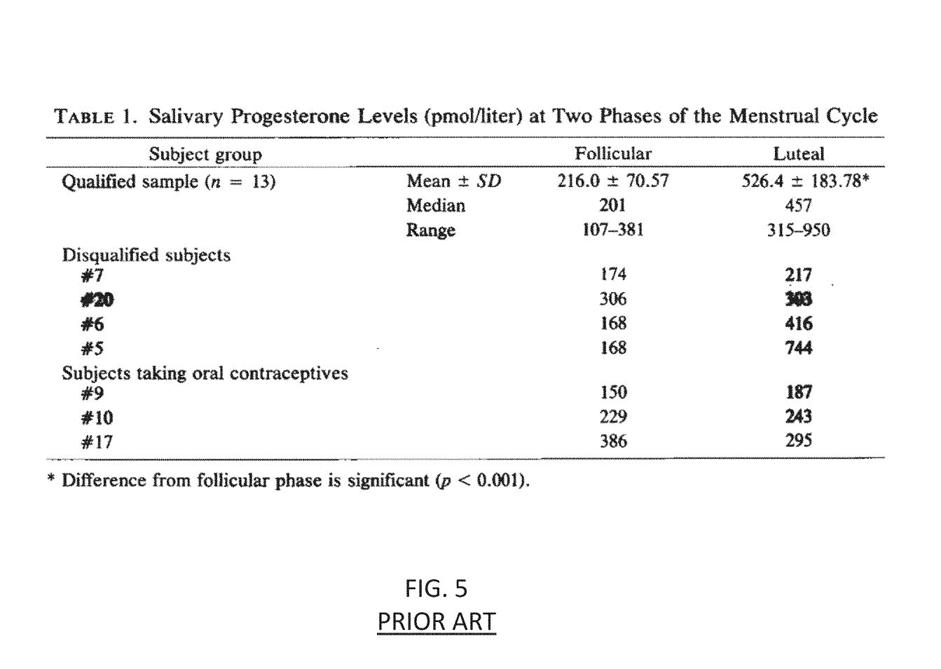

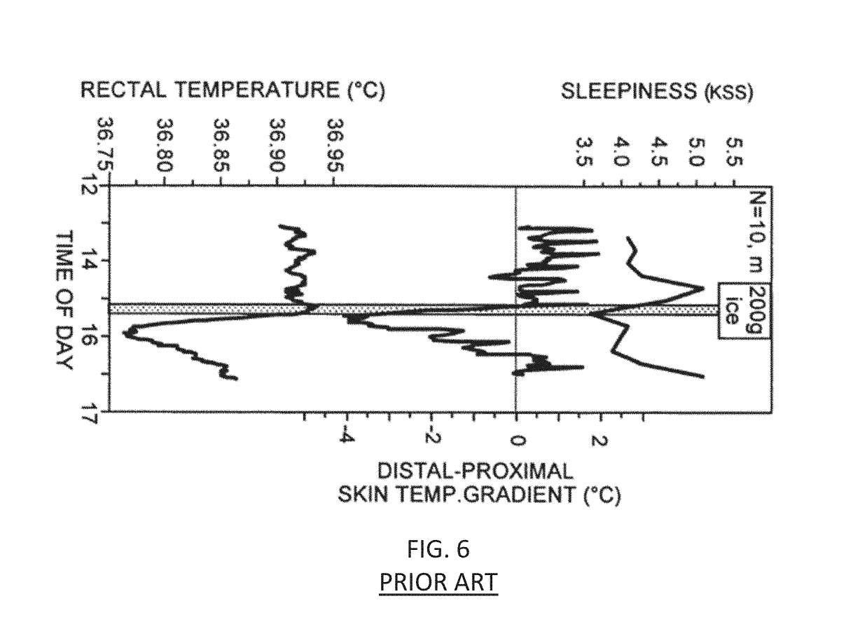

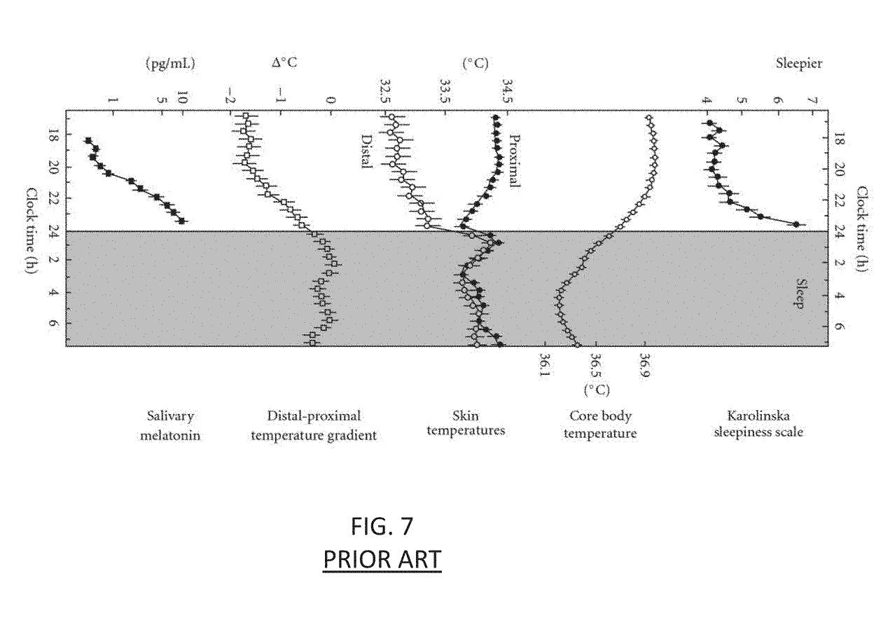

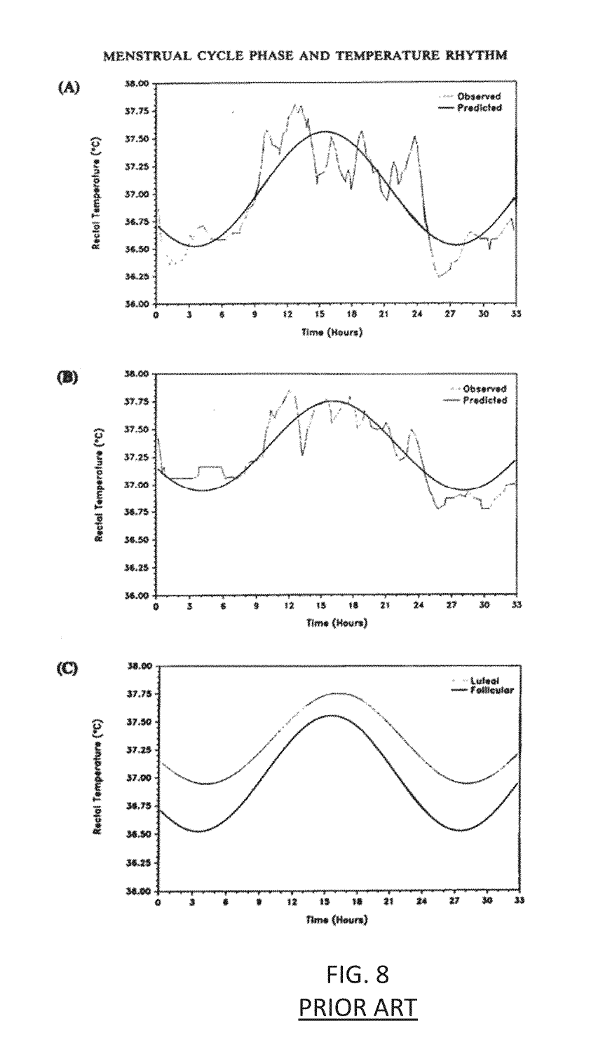

The detailed description explains embodiments of the disclosure, together with advantages and features, by way of example with reference to the drawings.

### DETAILED DESCRIPTION OF THE DISCLOSURE

As described herein, bioinformatics is combined with temperature data points to create a sophisticated analysis of the thermal state of a subject. As used herein, “subject”, or “user” means a human or other animal that has skin or other outer membranes that allows contact with one or more temperature sensors.

The method described here uses not only the values measured from the temperature sensors, but also uses information based on sensor behavior (e.g., component specific behavior such as sensitivity, resolution, calibration behavior and needs, drift, stability, power requirements, overheating, measurement changes resulting from usage or external factors, sample rate, battery life and other factors that are component specific), as well as information about circadian thermoregulation, core body temperature calculations based on distal and proximal temperature gradients, among other factors, as described further herein, to detect temperature change trends and analyze the data to obtain a physiological function result. The method described here is an accurate, comfortable, non-invasive, and unobtrusive method for body temperature tracking.

Through collecting body temperature data over time and analyzing it using bioinformatics or applied mathematics, as described herein, information related to a health condition, such as fertility level, kidney function, infection, fever, or other condition, can be determined. Data points that are considered outliers can be eliminated from the analysis, as is known in the art. Also, the analysis can add sophisticated models that takes other factors into account, such as those described herein.

In an embodiment, a physiological function result is determined using the method. A physiological function result can be an estimation of the core body temperature, a fertility parameter, a relative health parameter, or a metabolic function parameter, for example.

In one embodiment, two or more temperature sensors are placed on the skin of a subject at different locations. An additional one or more temperature sensors can be placed in such a way that they can be used to estimate ambient temperature.

The temperature sensors may be part of the same device or separate devices where their data can be collected simultaneously or sequentially and analyzed together or separately. The temperature sensors can be used to approximate a body temperature. The body temperature can be a core body temperature, a regional body temperature, or a peripheral body temperature. Regional body temperature can be a body temperature of the lower torso, for example. Peripheral body temperature can be a temperature of a peripheral body portion, such as a segmented body region, such as the head, left hand, right foot, etc. As will be appreciated by the description herein, the terms used for body temperature can overlap, and unless it is specified from the context, the terms are intended to be inclusive of each other. The temperature values can be fed through a software program where additional parameters can be included and other trends can be used in the prediction of body temperature, for example, the distal to proximal gradient, Newton's Law of Cooling, Circadian Thermoregulation, and other factors, as described herein.

Body temperature variability is complex and non-linear and can be influenced by multiple exogenous and endogenous factors. Studies have shown there is a correlation between body temperature variability and health. Without the ability to accurately identify body temperature non-invasively, it is difficult to capture data to understand the temperature patterns of individuals in healthy and diseased states. Body temperature measurements on their own are also not enough to understand the condition of an individual.

By collecting body temperature measurements on an ongoing basis, instead of an isolated temperature measurement, and by applying cyclical patterns to understand the data, including chronobiological patterns, and comparing the results to determine synchronization or de-synchronization and normal or abnormal states using mathematical models, one can determine the individual's health, predict disease or other conditions and their stage. For example, brain lesions, chronic diseases such as HIV, AIDS, cancers, insomnia, febrile states, depression, psychological or neurological states, anxiety, concussion, head trauma, allergies, and thyroid disorders can alter the temperature patterns and create abnormalities or de-synchronization. By looking at changes such as amplitude, mesor, mean, peak, nadirs, or acrophase, or a combination comprising one or more of the foregoing, one can determine the relative health and physical fitness level of an individual. For example, a healthy profile involves large temperature amplitudes.

Factors such as age, gender, physical fitness, height, weight, fat percentage, body mass index, lean body mass, body size proportions, site of measurement, and time of day can have major implications for conditions that the temperature is being measured for. For example, slight elevations in temperature for elderly patients may be a sign that there is a serious underlying cause. This same elevation in temperature in a younger patient may not be cause for alarm, but given a typical decreased ability to generate body heat in elderly patients this should not be dismissed. Additionally, the time of day is important since temperature fluctuations occur naturally throughout the day. Without correcting for time of day, it can be difficult to distinguish between what is a healthy and unhealthy temperature leading to improper diagnoses. BBT is known to have a 20% inaccuracy rate when pure BBT temperature measurements are used alone. This is because day-to-day variability of measurements, and other factors mentioned here can influence readings.

A number of masking effects and entraining agents can mask the rhythms and their impact on temperature patterns. These masking effects can include the use of drugs, especially those that influence hormones, such as contraceptives and estrogen replacement therapies, alcohol, and other factors such as clothing, meal timing, diet, lifestyle, caloric restriction, light-dark exposure, sleep deprivation, time zone, climate, and waking time, abrupt shifts in sleep and waking times, time zone, light-dark exposures, etc., among others, which have all been shown to impact temperature patterns, and at times result in de-synchronization.

By collecting periodic data over time, one can better understand temperature variability to identify abnormalities and de-synchronization. This information can be used to better understand temperature measurements, identify healthy and diseased states, and the effects of illness, medication, diet, and lifestyle on chronobiological processes.

The described method uses a predetermined prediction equation to obtain a physiological function result. The predetermined prediction equation uses as inputs the temperature values and determines a physiological function result. The temperature values used can include the amplitude of the temperature values, the mesor of the temperature values, the mean of the temperature values, the peak of the temperature values, the nadir of the temperature values, the acrophase of the temperature values, or a combination comprising one or more of the foregoing. The predetermined prediction equation is located in a memory, in an embodiment. The predetermined prediction equation can be modified, as known in the art. The predetermined prediction equation can compare changes in amplitude of the temperature values over time, for example. The predetermined prediction equation can further comprise a cyclic rhythmic parameter, such as a circadian rhythm, sleep-wake cycles, activity rhythms, circannual rhythm, circa mensal rhythm, or a combination comprising one or more of the foregoing, in the determination of a physiological function result. The predetermined prediction equation can further comprise masking parameters, entraining agent parameters, or both, in the determination of a physiological function result. Masking parameters or entraining agent parameters can include external factors, environmental, diet, lifestyle, clothing, drug consumption such as contraceptives, estrogen replacement therapy, liquid consumption such as alcohol consumption, meal timing, caloric restriction, sleep habits, light exposure, waking time, climate, time zone, schedule shifts, and temperature findings in diseased states. The predetermined prediction equation can further comprise temperature changes in thermogenic processes, such as the thermogenic properties of progesterone, in the determination of a physiological function result. The predetermined prediction equation can further comprise a personal parameter, wherein the personal parameter comprises age, gender, height, weight, fat percentage, body mass index, lean body mass, body size proportions, menstrual cycle day, chronotype, drug use (e.g. hormonal contraceptive), time zone, or a combination comprising one or more of the foregoing, in the determination of a physiological function result. The predetermined prediction equation can further comprise one or more of the following variables can be included in the predetermined prediction equation: time of day, time of year, physical activity, sleep deprivation, circadian rhythms, dietary factors, alcohol consumption, lighting conditions (e.g., ultraviolet (UV) index value or brightness), or a combination comprising one or more of the foregoing in the determination of a physiological function result.

The physiological function result can be a fertility parameter, relative health parameter, heat balance parameter, heat distribution parameter, rhythm strength parameter, or a metabolic function parameter. The physiological function result can be the determination of if the subject is in a thermoneutral zone. The physiological function result can be a fertility parameter, such as a level of fertility. The physiological function result can be a metabolic function parameter, such as an infection, or the presence of a fever. The physiological function result can be a metabolic function parameter such as thyroid function, cholesterol amount, pituitary function, gonadal function, adrenal function, hypoglycemia, pancreatitis, drug or alcohol abuse, kidney function, liver function, cardiovascular function, central nervous system function, metabolic toxicities, reproductive stage, fibromyalgia, or a combination comprising one or more of the foregoing. The physiological function result can be a determination of whether the subject is in a synchronized or desynchronized state in relation to chronobiological rhythms The physiological function result can be a determination of a health state, such as a normal, abnormal, or diseased state. The health state can be an indicator of abnormalities, such as brain lesions, cancer progression, Human Immunodeficiency Virus (HIV) acquired immune syndrome (AIDS) progression, cancer treatment efficacy, allergies, depression, thyroid imbalances, or febrile state.

In an embodiment, the physiological function result can be an indicator of psychological or neurological states, including emotional state, anxiety, truthfulness, excitation, depression, concussion, or head trauma, for example.

In an embodiment, the physiological function result further comprises comparison of a change in amplitude of the temperature values compared to historical data or cyclic patterns based on historical data or population data, to determine a physiological function result. The predetermined prediction equation can include other vital signs, such as heart rate or oxygenation level, in determining a physiological function result.

The temperature values can be obtained one time, more than one time, the same or similar time every day, or every hour, for example, or any other suitable time frame.

In an embodiment, a multiple-regression procedure is used in the predetermined prediction equation to calculate and describe any one or more of four circadian rhythm parameters: rhythm strength, mesor, acrophase, and amplitude. In an embodiment, bioinformatics is used, for example, to gather, store, analyze, and determine results using the temperature values obtained from a subject and the other information used, as described herein.

The bioinformatics can look for differences in mesor between phases and cycle events, for example. Peak temperature of a subject can also be analyzed. Studies have shown the peak temperature occurs in ovulating women at around 15 hours in a 24 hour period where 0 is midnight, the mean acrophase occurs at approximately 1530 hours, regardless of menstrual cycle phase. Using temperature data collected over time from a subject and the information described above regarding peak temperature and mean acrophase, for example, the predetermined prediction equation can determine when a woman is ovulating, for example.

Dampened temperature mesor, acrophase, rhythm strength, and/or amplitude can also be used to diagnose or report on disease, as well as assess fertility level/menstrual phase, or metabolic function. Differences occur between several healthy and unhealthy populations in circadian rhythm amplitudes, for example. For example, elderly men, advanced cancer patients, and women in the luteal phase show dampened amplitudes in comparison to healthy young men and women during the pre-ovulatory phase. Similarly to the determination of ovulation described above, the temperature amplitude values obtained can be used in the predetermined prediction equation with the amplitude information described above to obtain a physiological function result which is a fertility parameter or a metabolic function parameter, such as a thyroid function, cholesterol amount, pituitary function, gonadal function, adrenal function, hypoglycemia, pancreatitis, drug or alcohol abuse, kidney function, liver function, cardiovascular function, central nervous system function, metabolic toxicities, reproductive stage, or presence of fibromyalgia. Obtaining temperature values over time, such as every 5 minutes, every hour, every day, and at other time durations can be used to determine the subject's normal range. In addition, population data obtained by aggregating data from different subjects can be used to determine expected ranges for healthy and unhealthy populations. This information can be used in the predetermined prediction equation as a factor to determine a physiological function parameter for a subject.

Provided is also a method to create a thermal mapping of the body, comprising: obtaining temperature readings from one or more temperature monitors on one or more locations of a subject; inputting the temperature readings and locations into a processing unit configured to receive and process signals produced by the temperature monitors and an optional ambient environmental monitor. The thermal mapping can be a static map of one time point, or can be dynamic and change based on the changes of the temperature data over time. The thermal mapping can be compared to other thermal mappings to yield information regarding any of the states described herein, such as the body temperature, heat balance, physiological function, thermoregulation, or metabolic function states.

Provided is also a method to identify the placement site of sensors on the subject comprising: a processing unit configured to receive and process a user input identifying sensor placement such as part of body, side of body etc.; a camera on an external device utilizing feature recognition or signal detection from the on-subject device; triangulation between an external device and sensor device; a position indicator on an external device and a position indicator on one or more on-subject devices, or a combination comprising one or more of the foregoing.

A method to identify the placement site of sensors on the subject comprising: determination of the site based on sensor measurements, which can be correlated with measurements on a thermal mapping, is also provided.

A method to instruct a user on a particular placement of sensors on specific positions on the body, such as on the periphery (e.g. head, feet, hands), core (e.g., trunk), or other location, where the sensor can have an identifier (e.g. head sensor, foot sensor), or the user inputs identifying information for the sensor to an external device via an identification system providing a number, color code or other identifier, is also provided.

In an embodiment, an indicator that the wearer is in a state of rest or approaching sleep may be used and the information incorporated into calculations. For example, an accelerometer, gyroscope, or heart rate monitor or a combination may be used. The use of an accelerometer, gyroscope, or heart rate monitor to detect rest or approaching sleep is known in the art. The accelerometer, gyroscope, or heart rate monitor can be incorporated as part of the temperature sensor, or they can be stand-alone devices. In another embodiment the “activation” of the patch may be used an indicator that the subject is approaching a state of rest, by attaching the sensor to the body, activating the sensor via an application or switch, or other indicator that the sensor should commence measurement.

The skin temperature sensor can be any of a number of designs, such as a temperature sensor located on a membrane that can adhere to a subject's body. The sensors may be a sensor similar to that described in United States Patent Publication 2013/0197319 entitled “Flexible Electrode for Detecting Changes in Sweat,” the contents of which are incorporated herein by reference. The communications system can be as described in WO2015/143259, the contents of which are incorporated herein by reference. The actual temperature sensing part of the temperature sensor can be a flexible temperature sensor arrays using integrated circuits, which are metal thin-film interconnects and traces sandwiched between semi rigid polymer sheets; a conductive polymer composite; thin metal films; temperature sensitive fibers using polymer composites or carbon nanotubes; flexible temperature sensitive fabric, or any other suitable temperature sensing material or device.

The sensor can transmit the measurements from temperature monitors to an external computing device that can combine these measurements with other data from the subject and other information, as described above, such as that contained in clinical databases to estimate a core body temperature. This core body temperature can be used as described herein to determine a health state for a subject. It is not required that a core body temperature is estimated, rather, a physiological function result can be determined without the estimation of the core body temperature.

The sensor can receive a signal back from the external computing device and communicate a message to the subject or another person, such as a physician, via a display, an indicator, an audible tone, or a combination of the foregoing. In an embodiment, a controller cooperates with a processor on an external device, such as a cellular phone or a laptop computer for example, to determine a health state for a subject without transmitting data to a remote computing device. In an exemplary embodiment, a device communicates with a local electronic device, such as a cellular phone for example, that provides additional functionality to the subject or another user. In other embodiments, the device may communicate with other local external devices, such as but not limited to, a wearable device (e.g., a watch, fitness monitor) or another computing device (e.g., a desktop computer, a laptop, a tablet, appliances, or a television) for example. The external wearable device may be for example, glasses having a display that shows the subject the data or information from the device as described here. The wearable device may also be a watch with a display that shows the subject data or information from the device. The wearable device may further be an article such as a ring, broach, or pendant, that displays information from the device. It should be appreciated that these wearable devices may also indicate or display a subset of the data/information, for example, a ring may have an indicator that changes color based on the subject's fertility level. The wearable device and other external computing devices each have a processor and memory that is configured to execute computer instructions on the respective processor to perform the functions described herein.

In one embodiment, the cellular phone, wearable device and external computing devices may further have one or more applications or control methods that may remind the subject (such as by displaying data or information on a screen, actuating a visual indicator or the like) to use the device in the event that data is not received from the device, such as on a predetermined schedule for example.

In still other embodiments, the device, the cellular phone, or the wearable device may cooperate to provide additional information. For example, in one embodiment, the subject may provide information from another source, such as through the use or wearing of a fitness or activity monitor that measures subject parameters such as sensor and the temperature. As used herein the term “data model” includes any known data model for analyzing measured parameters, such as but not limited to a mathematical representation of collected data against which measured values may be compared. The data model may be in the form of a mathematical expression such as a line (e.g. a polynomial curve) fitted to the data or may be a database of values for example. As used herein the term “population data model” is a mathematical representation of collected data for a large group of individuals. In one embodiment, the population data model may be trained over time as additional subject data is collected and stored. In one embodiment, the data model may be trained using other data such as from either other demographic groups or a historical data that is directed at different demographic groups to prepopulate the data model to provide the desired accuracy level prior to the availability of large amounts of subject data. In one embodiment, the population data model may represent an average value for the group measured. In one embodiment, the population data model may represent a target demographic of individuals, where the collected data is segregated by age. The population or demographic data model may also represent expected temperature values for a normal healthy individual. As used herein, the term “personal data model” is a mathematical representation of historical data for the particular subject wearing the device. The temperature values measured by the device may be compared to both the population data model and the personal data model when determining a physiological function result. In embodiments where multiple subjects use the same device, the device may be configured to determine a personal data model based on the subject inputting a PIN or password.

The comparison of temperature values to data models may include comparing absolute temperature values or a relative change in the temperature values, or other aspects of the temperature values, as described herein. This can include comparisons in relation to their expected values and data models, as well as each other's values and data models. In one embodiment, the comparison of temperature values to the data models includes comparing a profile trend of temperature values measured over a plurality of time periods, such as over the course of several days for example, with the data model. The comparison of the profile trend may include profiles such as a rapid increase in BBT followed by a rapid decline in measured values for example, or may include a rapid increase in skin thickness, or heart rate, for example.

In one embodiment, the measured values are stored in memory and the personal data model is updated to include these stored values. The updating of the personal data model may be on a periodic or aperiodic basis. In one embodiment, the personal data model is updated in real time. As discussed herein, the measured values may be stored and combined with those from other individuals and incorporated into the population data model.

If the temperature sensors are each contained on the same device then there may be an indicator to the subject regarding how to place the device on the body, e.g. arrow indicating up or down. This is helpful in determining which temperature sensor represents a distal or proximal temperature in relation to the other sensor. If the sensors are located on a different device, there can be an indicator as to which device should be placed on the trunk (proximal) and limbs (distal) to represent the distal-proximal differences.

In one embodiment, the hardware can include an analog front end consisting of an ultra-low input bias current op-amp. This can feed a 12-bit analog-to-digital conversion (ADC), which can be controlled and read by a Bluetooth system on chip (Sock) (e.g. IEEE 802.15) IC (system on chip (SoC)). In one embodiment, an external ADC can be used to give the most flexibility and resolution for development. An ADC read can be done using the SoC's onboard peripheral.

In one embodiment, the ADC can have a built-in temperature sensor with a ⅛ degree centigrade resolution, in addition to 4 single-ended voltage inputs. If higher resolution is desired than that one eighth (⅛th) of a degree, temperature dependent resistors located at various places in the device can be read.

In one embodiment, three temperature sensors are used to gather temperature data simultaneously, or relatively simultaneously, to take a snapshot of a subject's current temperature profile: e.g. two temperature sensors next to the body to collect skin temperature readings, and another on the air-facing side of the device to sense closer-to-ambient temperature. The difference (delta) between the two inner temperatures can be used to determine the distal-proximal temperature gradient, while the delta between outer temperature and inner temperatures can be used to determine how ambient temperature is affecting the measurements.

In an embodiment, the Bluetooth IC runs firmware that collects data at a configurable rate. In one embodiment, this is set to a 5-second sample rate, but it could be any rate such as 5 minutes or more between measurements to conserve power. In one embodiment, the analog circuitry can be put into a standby mode (or shut off entirely) when a measurement is not being actively taken. In one embodiment, a flexible battery of the type that gets laminated into ISO standard plastic cards can be used. This flexible battery measures about 0.5 mm thick, which is allows packaging the device in a way that is unobtrusive to the subject.

The sizes and shapes of the devices are not limited to those specifically described here. Any number of suitable sizes and shapes can be used, as will be apparent to one of ordinary skill in the art. The power and transmission methods are not limited to those described here. Any number of other power and transmission methods and protocols can be used, as will be apparent to one of ordinary skill in the art.

If necessary for calibration, e.g., for use with fever tracking, it may be recommended that a subject takes his/her oral, forehead, or rectal temperature, and enter the value into the application prior to adhering the temperature patch.

For certain conditions, such as fertility tracking, it may be useful to detect changes in the data that are relative, e.g., amplitude modulation for different phases, and are not related to the exact temperature reading. For readings that are related to the exact temperature reading change, e.g., fever tracking or tracking during in or out-patient procedures, e.g. pre-operative procedures, an initial calibration based on an oral or rectal temperature reading, can allow for enhanced tracking and Core Body Temperature estimation with the system.

The described method uses relative temperature changes related to amplitude without exact temperature readings based on the exact degrees, and is therefore not reliant on being placed on the exact same spot each evening, for example. The described method is therefore not influenced by sleep stage and ambient temperature. In a particular embodiment, the described method reflects the thermogenic properties of progesterone that BBT devices are hoping to estimate. The described method therefore has advantages over BBT thermometers and BBT morning oral measurement devices, as well as standard temperature patches.

The accuracy of temperature recordings, for example, in detecting ovulation, may be enhanced by measurements taken just prior to the onset of sleep and evening temperatures, as well as morning temperatures for example, to determine the change in amplitude between follicular and luteal phases of the menstrual cycle. Studies have shown that a temperature recorded just prior to sleep onset, when thermoregulatory mechanisms are still intact, may be the most appropriate indicator of ovulation. In one study, the dampened circadian rhythm during the luteal phase appears to be primarily a function of a much higher increase in nocturnal T [temperature readings] in comparison with the smaller diurnal increase in T [temperature readings].

Statistical regression analyses have revealed that the distal-proximal temperature gradient (a measure of heat loss at the extremities) is the best predictor of a rapid SOL (Sleep Onset Latency).

In an embodiment, the data is used to generate a data model representing the core-to-peripheral redistribution of body heat and thermoregulatory vasomotion. This thermal mapping of the body can be based on individual and/or population data and parameters. This thermal mapping can include expected temperatures at different points on the body at different times. This heat map can be used as a parameter for analysis. The same thermal mapping can be dynamic and adjust according to a data model.

Another aspect of the method includes an ability to compensate for noise, measurement errors, fluctuations due to exogenous factors, and a calibration function. The described method allows for elimination of readings that do not correlate with the expected trends, especially in relation to cyclical data, and do a comparison over time.

In an embodiment, the sensor(s) are packaged in a patch or wearable. This makes the system attractive in terms of subject comfort. In one embodiment, the subject can wear a medium sized adhesive device, similar to the size of an adhesive bandage on his/her body—with the purpose of quickly forgetting they are wearing it.

The patches can be disposable where some or all of the patch will be disposed of after removal from the body. In one embodiment, the patches are worn for 1 night or 1 day, or up to 1 week, similar to the birth control patch with 4 in a monthly pack. In another embodiment the patch or parts of the patch are disposed of after one use. In one embodiment the device has the electronics (e.g., bluetooth transponder and analog front end) contained in one unit, which is configured to allow insertion of expendable components (such as sensors and battery) contained in a separate unit.

With this method the electronic footprint can be minimized, as well allowing flexible, ultra thin batteries to minimize total bulk and remove the need for connectors or additional hardware to recharge the battery. In one embodiment the power source can be an ultra thin battery from the company, Flexion—which is designed to be laminated into credit cards and measure ˜0.5 mm in thickness. In one embodiment, the device contains a rechargeable power source.

In one embodiment, the disposable patch can be built in such a way to hold the electronics within a pocket, adhere itself to the skin, and expose the sensors directly to the skin surface for measurement. This would mean the costs of replacing the entire patch and electronics for each use are de-coupled.

In one embodiment, the patch size is determined by the optimal distal-proximal temperature sensor placement. The patch will then have indicators as to top and bottom so the application can easily identify which temperature sensor refers to which measurement location.

In one embodiment, the data is collected and sent to an external device such as a mobile phone where the analysis is done. In one embodiment, some analysis is done on the device. In one embodiment, an indicator on the device shows the current health state (e.g. condition status, fertility level, temperature in degrees, warning indicators). In one embodiment, the changes in the health status or temperature status as a result of the values sensed can trigger a notification or change of an indicator on the sensing device or on an external device.

In one embodiment, the communication or transfer of information between the sensing device and the external device can be done via Bluetooth or RFID or other means of wireless communication. The communication or transfer of information between the sensing device and the external device can also be done via a wire.

This data can be collected over time to build a model that improves and learns to improve estimates regarding CBT, Fertility/Hormonal Level States, metabolic function, and other health conditions.

While devices in the form of a patch have been described, it should be appreciated that the device can take many different forms such as a wearable, a wearable attachment, an accessory (e.g. a piece of jewelry, a watch), etc. The device does not also have to be disposable.

In another embodiment, other rhythms may be used in the analysis of determining the health condition or fertility level, such as the lunar cycle and other natural rhythms, and the syncing or out of syncing of the individuals cycle, as well as sensing light patterns (in the form of an on-device light sensor or an external or third party light sensor, light data, lunar cycle data) that may be impacting the subject's state especially for women, patterns that may be influencing the menstrual cycle artificially or naturally. Studies have shown that women with PMDD (Pre-menstrual Dysphoric Disorder) exhibit abnormally shifted cycle phases and acrophases. When these women are exposed to light therapy in the evening, improvements regarding symptoms of PMDD as well as shifts in their cycle to more regular patterns have occurred.

The methods and systems are further illustrated by the following embodiments, which are non-limiting.

Embodiment 1: A method to determine a physiological function result, comprising: determining the temperature of a first body location on a subject to obtain a first body temperature value; optionally determining the temperature of a second body location on a subject to obtain a second body temperature value; optionally determining the ambient temperature to determine an ambient temperature value; obtaining a physiological function result from a predetermined prediction equation, wherein the obtaining comprises a comparison of the amplitude of the temperature values, the mesor of the temperature values, the mean of the temperature values, the nadir of the temperature values, the peak of the temperature values, or the acrophase of the temperature values, or a combination comprising one or more of the foregoing.

Embodiment 2: The method of Embodiment 1, wherein the obtaining comprises inputting the temperature values into the predetermined prediction equation.

Embodiment 3: The method of any of Embodiments 1-2, further comprising determining a temperature value at more than one time.

Embodiment 4: The method of any of Embodiments 1-3, wherein the predetermined prediction equation further comprises a comparison of changes of amplitude of the temperature values over time.

Embodiment 5: The method of any of Embodiments 1-4, wherein the predetermined prediction equation further comprises a cyclic rhythm parameter.

Embodiment 6: The method of any of Embodiments 1-5, wherein the cyclic rhythm parameter is a circadian rhythm, sleep-wake cycles, activity rhythms, circannual rhythm, circa mensal rhythm, or a combination comprising one or more of the foregoing.

Embodiment 7: The method of any of Embodiments 1-6, wherein the predetermined prediction equation further comprises masking parameters, entraining agent parameters, or both.

Embodiment 7A: The method of any of Embodiments 1-7, wherein masking parameters or entraining agent parameters include external factors, environmental, diet, lifestyle, clothing, drug consumption such as contraceptives, estrogen replacement therapy, liquid consumption such as alcohol consumption, meal timing, caloric restriction, sleep habits, light exposure, waking time, climate, time zone, schedule shifts, and temperature findings in diseased states.

Embodiment 8: The method of any of Embodiments 1-7A, wherein the predetermined prediction equation further comprises cyclical changes in thermogenic processes.

Embodiment 9: The method of any of Embodiments 1-8, wherein the predetermined prediction equation further comprises a personal parameter, wherein the personal parameter comprises age, gender, menstrual cycle day, height, weight, fat percentage, body mass index, lean body mass, body size proportions, chronotype, drug use (e.g. hormonal contraceptive), time zone, or a combination comprising one or more of the foregoing.

Embodiment 10: The method of any of Embodiments 1-9, wherein the physiological function result is a fertility parameter or a metabolic function parameter.

Embodiment 11: The method of any of Embodiments 1-10, wherein the physiological function result is a determination of if the subject is in a thermoneutral zone.

Embodiment 12: The method of any of Embodiments 1-11, wherein the fertility parameter is a level of fertility.

Embodiment 13: The method of any of Embodiments 1-12, wherein the metabolic function parameter is an infection, or the presence of a fever.

Embodiment 14: The method of any of Embodiments 1-13, wherein the metabolic function parameter is thyroid function, cholesterol amount, pituitary function, gonadal function, adrenal function, hypoglycemia, pancreatitis, drug or alcohol abuse, kidney function, liver function, cardiovascular function, central nervous system function, metabolic toxicities, reproductive stage, or fibromyalgia.

Embodiment 15: The method of any of Embodiments 1-14, wherein the physiological function result is a determination of whether the subject is in a synchronized or desynchronized state in relation to chronobiological rhythms

Embodiment 16: The method of any of Embodiments 1-15, wherein the physiological function result is a determination of a health state, such as a normal, abnormal, or diseased state.

Embodiment 17: The method of any of Embodiments 1-16, wherein the health state is an indicator of abnormalities, such as brain lesions, cancer progression, Human Immunodeficiency Virus (HIV) acquired immune syndrome (AIDS) progression, cancer treatment efficacy, allergies, depression, psychological or neurological states, concussion, head trauma, thyroid imbalances, organ failure, febrile state.

Embodiment 18: The method of any of Embodiments 1-17, wherein the temperature values are input into one or more mathematical models comprising historical or population data related to one or more of the following: healthy, abnormal, or diseased states based on individual data, historical data or population data.

Embodiment 19: The method of any of Embodiments 1-18, wherein the determining is performed periodically.

Embodiment 20: The method of any of Embodiments 1-19, wherein the first body location is distal to the core, and the second body location is proximal to the core and wherein the difference between the first and second temperature values is a first distal to proximal temperature gradient; the method further comprising: optionally repeating the determining steps to determine a second distal to proximal temperature gradient; inputting the first and second distal to proximal temperature gradient in a predetermined physiological function equation to obtain a physiological function result.

Embodiment 21: The method of any of Embodiments 1-20, further comprising inputting the first and second distal to proximal temperature gradient into a predetermined prediction equation to approximate a body temperature, such as the core body temperature, a regional body temperature, and/or peripheral body temperature(s)

Embodiment 22: The method of any of Embodiments 1-21, wherein the predetermined prediction equation further comprises the distance of the first or second body location to the core.

Embodiment 23: The method of any of Embodiments 1-22, wherein the predetermined prediction equation further comprises the distance between the first and second body location.

Embodiment 24: The method of any of Embodiments 1-23, wherein the predetermined prediction equation uses the change of amplitude of the first body temperature value and optionally the second body temperature value to determine a predicted core body temperature.

Embodiment 25: The method of any of Embodiments 1-24, wherein the predetermined prediction equation uses the change of amplitude of the second body temperature value to determine a predicted core body temperature.

Embodiment 26: The method of any of Embodiments 1-25, wherein the physiological function result further comprises comparison of a change in amplitude of the temperature values compared to historical data or cyclic patterns based on historical data or population data, to determine a physiological function result.

Embodiment 27: The method of any of Embodiments 1-26, further comprising determining the heart rate.

Embodiment 28: A system for determining a health state, comprising: two or more sensors, each sensor comprising: a membrane configured to be applied to an external surface of a body of a subject; one or more temperature monitors configured to detect and/or measure a temperature, each monitor located within the sensor in thermal contact with the membrane; optionally, a transmitting apparatus configured to transmit the output from the one or more temperature monitors; a power source configured to apply power to the one or more temperature sensors and the transmitting apparatus; optionally, an ambient environmental monitor configured to detect and/or measure an environmental condition in a vicinity of a subject; a processing unit configured to receive and process signals produced by the temperature monitors and optional ambient environmental monitor, to determine time-dependent parameters of temperature change, and to calculate a body temperature, such as the core body temperature, a regional body temperature, and/or peripheral body temperature(s).

Embodiment 29: The system of Embodiment 28, further comprising an indicator on the device that can be viewed or heard by the subject.

Embodiment 30: The system of any of Embodiments 28-29, wherein the processing unit is further configured to determine a physiological function result.

Embodiment 31: A kit for determining a health state, comprising: one or more sensors, each sensor comprising: a membrane configured to be applied to an external surface of a body of a subject; one or more temperature monitors configured to detect and/or measure a temperature, each monitor located within the sensor in thermal contact with the membrane; a transmitting apparatus configured to transmit the output from the one or more temperature monitors; a power source configured to apply power to the one or more temperature sensors and the transmitting apparatus; optionally, an ambient environmental monitor configured to detect and/or measure an environmental condition in a vicinity of a subject; instructions for using the one or more sensors, the instructions comprising access to a processing unit configured to receive and process signals produced by the temperature monitors and optional ambient environmental monitor, to determine time-dependent parameters of temperature change, and to calculate a core body temperature.

Embodiment 32: The kit of Embodiment 31, wherein the instructions further comprise access to a local or remote storage medium to obtain information about the health state.

Embodiment 33: The kit of any of Embodiments 31-32, wherein the local or remote storage medium comprises a predetermined prediction equation configured to obtain a physiological function result.

Embodiment 34: A method to identify the placement site of sensors on the body comprising: a user input to identify placement such as part of body, side of body; a camera on an external device utilizing feature recognition or one or more signal detection from the on-body device or a combination; triangulation between an external device and sensor device; a position indicator on an external device and a position indicator on one or more on body devices; placement of specific sensors according to instructions on different parts of the body, or a combination comprising one or more of the foregoing.

Embodiment 35: A method to calibrate the sensors using input of other physiological data.

Embodiment 36: The method of Embodiment 35, wherein the other physiological data comprises oral, forehead, ear, or rectal temperature measurements.

Embodiment 37: A method to create a thermal mapping of the body, comprising: obtaining temperature readings from one or more temperature monitors on one or more locations location of a subject; inputting the temperature readings and locations into a processing unit configured to receive and process signals produced by the temperature monitors and an optional ambient environmental monitor. The thermal mapping can be a static map of one time, or can be dynamic and change based on the changes of the temperature data over time.

Embodiment 38: A method to identify the placement site of sensors on the subject comprising: a processing unit configured to receive and process a user input identifying sensor placement such as part of body, side of body; a camera on an external device utilizing feature recognition or signal detection from the on-subject device; triangulation between an external device and sensor device; a position indicator on an external device and a position indicator on one or more on-subject devices, or a combination comprising one or more of the foregoing.

Embodiment 39: A method to identify the placement site of sensors on the subject comprising: determination of the site based on sensor measurements, which can be correlated with measurements on a thermal mapping.

Embodiment 40: A method to instruct a user on a particular placement of sensors on specific positions on the body, such as on the periphery (e.g. head, feet, hands), core (e.g., trunk), or other location, where the sensor may have an identifier (e.g. head sensor, foot sensor), or the user inputs identifying information for the sensor to an external device via an identification system providing a number, color code or other identifier.

The term “about” is intended to include the degree of error associated with measurement of the particular quantity based upon the equipment available at the time of filing the application. For example, “about” can include a range of ±8% or 5%, or 2% of a given value.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The terms “first,” “second,” and the like, “primary,” “secondary,” and the like, as used herein do not denote any order, quantity, or importance, but rather are used to distinguish one element from another.

While the disclosure is provided in detail in connection with only a limited number of embodiments, it should be readily understood that the disclosure is not limited to such disclosed embodiments. Rather, the disclosure can be modified to incorporate any number of variations, alterations, substitutions, or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

1. A method to determine a heat distribution, comprising:
determining the temperature of a first body location on a subject to obtain a first body temperature value;
determining the temperature of a second body location on a subject to obtain a second body temperature value;
applying differential analytics to build a model of heat distribution of the subject, wherein input to the differential analytics comprises the first body temperature value and the second body temperature value; and
determining a health condition of the subject based at least in part on the model of heat distribution of the subject.

2. The method of claim 1, further comprising determining a temperature value at more than one time.

3. The method of claim 1, wherein the health condition is further determined based at least in part on a cyclic rhythm parameter.

4. The method of claim 1, wherein the health condition comprises one or more of a thyroid function, cholesterol amount, pituitary function, gonadal function, adrenal function, hypoglycemia, pancreatitis, drug or alcohol abuse, kidney function, liver function, cardiovascular function, organ failure, thermoregulation function, heat balance, heat distribution, central nervous system function, metabolic toxicities, metabolic rate, metabolic function, reproductive stage, and fibromyalgia.

5. The method of claim 1, wherein the health condition is an indicator of abnormalities, the abnormalities comprising one or more of brain lesions, cancer progression, Human Immunodeficiency Virus (HIV) acquired immune syndrome (AIDS) progression, cancer treatment efficacy, allergies, depression, psychological or neurological states, anxiety, concussion, head trauma, thyroid imbalances, metabolic, and febrile state.

6. The method of clam 1, wherein the determining a health condition is further based at least in part on historical or population data related to one or more of the following: healthy, abnormal, or diseased states based on individual data, historical data or population data.

7. The method of claim 1, wherein the determining the temperature of a first body location on a subject and the determining the temperature of a second body location on a subject are performed periodically.

8. The method of claim 1 wherein the first body temperature is obtained by a temperature sensor located at the first body location on the subject, the temperature sensor comprising an accelerometer to determine the first body location of the subject.

9. The method of claim 1, further comprising:
determining an ambient temperature to determine an ambient temperature value, wherein the determining a health condition of the subject is further based at least in part on the ambient temperature value.

10. The method of claim 1, wherein the determining the temperature of a first body location, determining the temperature of a second body location, applying differential analytics to build a model of heat distribution of the subject, and determining a health condition of the subject are repeated on a periodic basis.

11. The method of claim 1, wherein the determining a health condition of the subject is further based at least in part on input of analyte values of the subject

12. The method of claim 1, wherein the determining a health condition of the subject is further based at least in part on whether the subject is in a synchronized or desynchronized state in relation to chronobiological rhythms of the subject

13. The method of claim 1, wherein a sweat rate of the subject is input to building a model of heat distribution of the subject.

14. The method of claim 1, wherein the humidity of the subject is input to building a model of heat distribution of the subject.

15. The method of claim 1, further comprising applying the differential analytics to build a second model of thermoregulatory vasomotion of the subject.

16. The method of claim 1, further comprising determining a treatment of the subject based at least in part on the health condition of the subject.

17. The method of claim 16, further comprising initiating the treatment of the subject.

18. A method to determine a biological process distribution, comprising:
determining a biological process marker of a first body location on a subject to obtain a first body biological process marker value;
determining a biological process marker of a second body location on a subject to obtain a second biological process marker value;
applying differential analytics to build a model of biological process distribution of the subject, wherein input to the differential analytics comprises the first body biological process marker value and the second body biological process marker value; and
determining a health condition of the subject based at least in part on the model of biological process distribution of the subject.

19. The method of claim 18, further comprising determining a treatment of the subject based at least in part on the health condition of the subject.

20. The method of claim 19, further comprising initiating the treatment of the subject.
